(19) United States
(12) Patent Application Publication
(10) Pub. No.: US 2022/0203039 A1
(43) Pub. Date: Jun. 30, 2022

(54) Rotation Sensing Arrangement for an Injection Device

(71) Applicant: Sanofi, Paris (FR)

(72) Inventor: Thomas Klemm, Frankfurt (DE)

(21) Appl. No.: 17/607,581

(22) PCT Filed: Apr. 29, 2020
(86) PCT No.: PCT/EP2020/061813

§ 371 Date: Oct. 29, 2021

(30) Foreign Application Priority Data
May 3, 2019 (EP) 19305567.0

(51) Int. Cl. A61M 5/315; G01D 5/20; G01D 5/24

(52) U.S. Cl. CPC A61M 5/31533; A61M 5/31551; G01D 5/204; A61M 2205/582; A61M 2205/0294; A61M 2205/3317; A61M 2205/581; G01D 5/2405

(57) ABSTRACT

The present disclosure relates to a rotation sensing arrangement for an injection device. The rotation sensing arrangement includes a first member and a second member that are rotatable relative to each other with regard to an axis of rotation, at least one signal generator arranged on the first member, at least one sensor arranged on the second member, and a processor (240) connected to the at least one sensor. The at least one sensor includes an interdigital electrode structure configured to generate an electrical signal in response to a movement of the at least one signal generator relative to the sensor. The processor is operable to calculate an angle of rotation of the first member relative to the second member on the basis of the electrical signal.

### CROSS REFERENCE TO RELATED APPLICATIONS

The present application is the national stage entry of International Patent Application No. PCT/EP2020/061813, filed on Apr. 29, 2020, and claims priority to Application No. EP 19305567.0, filed on May 3, 2019, the disclosures of which are incorporated herein by reference.

### TECHNICAL FIELD

The present disclosure relates to the field of rotation sensors, in particular to rotation sensors configured for detecting and/or quantitatively measuring a rotation of a component of an injection device. In one aspect the disclosure relates to a rotation sensing arrangement implemented in an add-on device configured for attachment to an injection device. In one aspect the disclosure relates to a rotation sensing arrangement implemented in an injection device. In a further aspect the disclosure relates to an injection device equipped with a rotation sensing arrangement configured to detect and/or configured to quantitatively measure a rotation of the component of the injection device. In a further aspect the disclosure relates to a method of determining and/or quantitatively measuring a rotation of a component of an injection device.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors have to meet a number of user-specific requirements. For instance, with patient's suffering chronic diseases, such like diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size.

Moreover, a dose setting as well as a dose dispensing procedure must be easy to operate and has to be unambiguous.

Typically, such devices comprise a housing or a particular cartridge holder, adapted to receive a cartridge at least partially filled with the medicament to be dispensed. The device further comprises a drive mechanism, usually having a displaceable piston rod to operably engage with a bung or piston of the cartridge. By means of the drive mechanism and its piston rod, the bung or piston of the cartridge is displaceable in a distal or dispensing direction and may therefore expel a predefined amount of the medicament via a piercing assembly, e.g. in form of an injection needle, which is to be releasably coupled with a distal end section of the housing of the drug delivery device.

The medicament to be dispensed by the drug delivery device may be provided and contained in a multi-dose cartridge. Such cartridges typically comprise a vitreous barrel sealed in distal direction by means of a pierceable seal and being further sealed in proximal direction by the bung. With reusable drug delivery devices an empty cartridge is replaceable by a new one. In contrast to that, drug delivery devices of disposable type are to be entirely discarded when the medicament in the cartridge has been dispensed or used-up.

With some drug delivery devices, such as pen-type injection devices a user has to set a dose of equal or variable size by rotating a dose dial in a clockwise or dose-incrementing direction relative to a body or housing of the injection device. For injecting and expelling of a dose of a liquid medicament the user has to depress a trigger or dose button in a distal direction and hence towards the body or housing of the injection device. Typically, the user uses his thumb for exerting a distally directed pressure onto the dose button, which is located at a proximal end of the dose dial and the dose dial sleeve, while holding the housing of the injection device with the remaining fingers of the same hand.

For mechanically implemented injection devices it is desirable to enable a precise, reliable and quasi-automated supervision and/or collection of injection-related data during use of the injection device. Mechanically operated injection devices may be equipped with an electronically implemented add-on device or data collection device configured to monitor user-induced operation of the injection device. A data collection device for attachment to an injection device should be rather compact with regards to its geometric size. For data collection devices configured for attachment to mechanically implemented injection devices it is a challenge to detect and/or to quantitatively measure the manual operation of the device conducted by the user of the device, e.g. when a user rotates a dial member of the injection device during setting of a dose or when a rotatable component of the injection device is subject to rotation during expelling of a dose

But also with electronically implemented injection devices, e.g. injection device equipped with an electric drive it is desirable to provide a precise, reliable and failure safe quantitative measuring of rotatable components of the injection device.

### SUMMARY

This disclosure relates to an improved rotation sensing arrangement configured to detect and/or to quantitatively measure a rotation of a rotatable component of an injection device. The rotation sensing arrangement and the sensor principle may be universally applicable to injection devices as well as to add-on devices configured for attachment to such injection devices.

The rotation sensing arrangement may be generally applicable to a variety of different rotatable components of an injection device or of an add-on device. The rotation sensing arrangement can be cost efficient to manufacture and can feature a rather compact design and geometry. The rotation sensing arrangement can be rather robust. In some aspects it provides and enables a contactless sensing and/or quantitative measuring of a rotation between at least a first member and a second member. The rotation sensing arrangement may be easily implementable in electronic devices and should be easily integratable into printed circuit boards.

In one aspect there is provided a rotation sensing arrangement for an injection device. The rotation sensing arrangement comprises a first member and a second member. The first member and the second member are rotatable relative to each other with regard to an axis of rotation. At least one of the first member and the second member is rotatable relative to the other one of the first member and the second member. For instance, the first member can be a stationary member or a stationary component of the injection device or of an add-on device whereas the second member is rotatable relative to the first member. With other implementations, it is the second member that is stationary whereas the first member is rotatable relative to the second member and/or relative to a housing of the injection device or of the add-on device.

The rotation sensing arrangement further comprises at least one signal generator that is arranged on or attached to the first member. Typically, the at least one signal generator is located on a particular portion of the first member. Typically, the at least one signal generator is arranged off axis, typically at a given radial distance from the axis of rotation.

The rotation sensing arrangement further comprises at least one sensor. The sensor is arranged on or attached to the second member. The at least one sensor comprises an interdigital electrode structure. The interdigital electrode structure is configured to generate an electrical signal in response to a movement of the at least one signal generator relative to the sensor.

The rotation sensing arrangement further comprises a processor connected to the at least one sensor. The processor is operable or configured to calculate an angle of rotation of the first member relative to the second member on the basis of the electrical signal. The electrical signal that is processed by the processor is typically obtained from the at least one sensor, in particular from the interdigital electrode structure of the at least one sensor. With some examples, the interdigital electrode structure is directly connected to the processor in a signal transferring way. In this way and as soon as the interdigital electrode structure generates a measurable signal it can be immediately processed by the processor to calculate or to derive a degree or rotation of the first member relative to the second member.

Typically and with some examples the first member and the second member each comprise an axis of symmetry substantially coinciding with the axis of rotation. The first member and the second member can be arranged at an axial offset with regard to each other. Here, the axial direction is parallel to or coincides with the axis of rotation. The first member may comprise an axial surface, e.g. a proximal or a distal surface facing towards a corresponding axial face of the second member. Likewise, the second member may comprise an axial face facing towards the first member. Typically, the at least one signal generator is arranged on a face or on a side of the first member facing towards the second member. Likewise, the at least one sensor of the second member is typically located or arranged on a face or on a side of the second member facing towards the first member.

With some examples and when the first member and the second member are arranged at a predefined axial offset from each other, the radial and/or circumferential extent of the first member substantially overlaps in axial direction with the respective radial and/or circumferential extent of the second member; and vice versa.

With other examples the first member and the second member substantially overlap in axial direction. Hence, with regard to the axial direction the first member and the second member may be located in a common plane transverse to the axial direction as defined by the axis of rotation. Here, the first member and the second member are located at a radial and/or circumferential offset relative to each other. As an example, at least a portion of the first member can be located radially inside a portion of the second member. Likewise, a portion of the second member can be located radially inside the first member. Here, the signal generator is arranged at a radial distance from the at least one sensor.

With numerous examples and as the first member is subject to a rotation relative to the second member with regard to the axis of rotation the at least one signal generator passes by the at least one sensor thereby inducing a measurable electrical signal in the interdigital electrode structure.

This electrical signal can be processed by the processor. Since the position of the at least one signal generator on the first member and the position of the at least one sensor on the second member is known the measurable generation of the electric signal by the interdigital electrode structure is hence indicative that the at least one signal generator has passed by the at least one sensor and/or its interdigital electrode structure.

Every time the at least one signal generator passes by the at least one sensor a respective electric signal is generated. Each occurrence of the signal generator passing by the sensor corresponds to a predefined angular distance of the rotation of the first member relative to the second member. By counting the number of electric signal generated over time, the processor is enabled to derive a total angular displacement or rotation of the first member relative to the second member.

Providing the at least one sensor with an interdigital electrode structure allows to miniaturize the rotation sensing arrangement. An interdigital electrode structure only requires a minimum of construction space on the second member. Moreover, interdigital electrode structures can be easily manufactured, e.g. printed or coated on the second member or on a respective substrate, thus enabling a low-cost mass-manufacturing of rotation sensing arrangements for injection devices.

This low-cost approach and by implementing interdigital electrode structures as a sensing component of the at least one sensor enables an integration of an electronic rotation sensing arrangement even into disposable injection devices. However, the presently proposed rotation sensing arrangement is not limited to disposable injection devices. It can be equally used for reusable injection devices as well as for add-on devices configured and intended for mechanical coupling with an injection device.

According to a further example the rotation sensing arrangement comprises a planar substrate. The planar substrate is typically located on the second member. The at least one sensor is arranged on the planar substrate. The entirety or only components of the at least one sensor can be arranged on the planar substrate. A planar substrate is of particular use for a cost-efficient and hence low-cost mass-manufacturing of electronic components. Providing of a planar substrate is of particular use for the implementation of the interdigital electrode structure. The interdigital electrode structure can be rather easily mounted and/or arranged and/or fixed on such a planar substrate.

The planar substrate may be a substrate separate from the second member. With other examples the planar substrate and the second member may be integrated. Hence, the second member may form, constitute or provide the planar substrate. In particular, one side or axial face of the second member may serve as a planar substrate on which the at least one sensor is directly arranged.

According to a further example the interdigital electrode structure is printed or coated on the planar substrate. A printing or coating of the electrically conductive interdigital electrode structure on the planar substrate is of particular use for providing a low-cost rotation sensing arrangement suitable for a mass-manufacturing process. Printing or coating the interdigital electrode structure directly on the planar substrate is of particular use from a manufacturing point of view. Hence, a separate assembly step of attaching the interdigital electrode to the substrate can be avoided. Respective costs and expenditure for a manual or mechanical assembly can be saved. Moreover, by printing or coating the interdigital electrode structure directly on the planar substrate a long-lasting and rather robust connection between the interdigital electrode and the planar substrate can be provided. This is of particular benefit for the lifetime, the robustness and the reliability of the rotation sensing arrangement.

According to a further example the rotation sensing arrangement comprises a printed circuit board. The interdigital electrode structure of the at least one sensor is arranged on the printed circuit board. The processor of the rotation sensing arrangement is also arranged on the printed circuit board. Moreover, the planar substrate as described above may be integrated into or on the printed circuit board. Hence, the planar substrate may be formed or constituted by the printed circuit board. In this way, the processor and/or further electronic components of the rotation sensing arrangement as well as the interdigital electrode structure can be arranged, mounted and/or fixed on one and the same printed circuit board. Moreover, all electrically conductive components of the rotation sensing arrangement can be mounted, arranged, printed or coated on one and the same planar substrate, which may be implemented as a printed circuit board. In this way and for manufacturing of the rotation sensing arrangement it may be sufficient to adequately configure a single printed circuit board and to arrange the printed circuit board on the second member.

The printed circuit board may be further provided with an electric source of energy, e.g. in form of a battery or solar cell. Typically, the at least one sensor and the processor are located on one and the same side of the printed circuit board. The battery or the electric energy supply may be located on an opposite side of the printed circuit board. It may be provided on a backside of the printed circuit board. The battery and/or the electric source of energy can be both attached and fixed to opposite sides of the printed circuit board.

The at least one sensor and the processor are typically located on one and the same side of the printed circuit board. With some examples the at least one sensor and the processor are located on opposite sides of the printed circuit board or of the planar substrate.

In this way the overall geometry of the rotation sensing arrangement can be modified and adapted in accordance to the available construction space inside the injection device and/or inside the add-on device.

According to a further example the interdigital electrode structure is configured to generate an electric field. The at least one signal generator is configured to modify the electric field. The interdigital electrode structure may form an interdigital capacitor having at least a first electrode and a second electrode. The first electrode and the second electrode are typically electrically insulated from each other.

The interdigital electrode structure can be driven by a DC voltage in a quasi-steady state configuration. The interdigital electrode structure can also be driven by an AC voltage. With each case a respective electric field will be generated between the first and the second electrodes being of different polarity. The at least one signal generator is configured to modify the electric field generated by the interdigital electrode structure. By bringing the at least one signal generator in close vicinity to the interdigital electrode structure, e.g. when passing by the interdigital electrode structure the at least one signal generator induces a measurable modification of the electric field. This signal generator-induced modification of the electric field is detectable by the processor connected to the at least one sensor and/or to the interdigital electrode structure. In this way, a counting pulse indicative of the at least one generator passing by the interdigital electrode structure or passing by the at least one sensor can be detected and acquired.

According to a further example the interdigital electrode structure comprises a first electrode and a second electrode. The first electrode and the second electrode are arranged in an interleaved geometric configuration. The first and second electrodes may comprise a periodic microstrip electrode structure with an interdigital pattern. The first and second electrodes may each comprise a comb-like structure, wherein the free ends of the comb-like structures face to each other and intersect each other contactlessly while being arranged in a common plane on the substrate. The first and the second electrodes may be also arranged in a meander-like way. In any of the conceivable interleaved geometric configurations of first and second electrodes the surface density of the electrode structure on the substrate can be increased or even maximized.

With a further example of the rotation sensing arrangement the interdigital electrode structure comprises a first electrode and a second electrode, wherein the first electrode and the second electrode are mutually electrically connected by a meandering conductive structure. Here, the first electrode and the second electrode may be implemented as contact terminals of a single electrically conductive structure, wherein the electrically conductive structure is of meandering type or comprises a meandering geometry.

A meandering conductive structure may comprise numerous elongated conductor sections extending parallel to each other. The numerous elongated conductor sections are electrically connected in line. With an example of at least three elongated conductor sections, a first longitudinal end of the first conductor section may constitute the first electrode or may be connected to the first electrode. An opposite second longitudinal end of the first conductor section may be connected to a second longitudinal end of a neighboring second conductor section.

An opposite end of the second conductor section, hence the first end of the second conductor section, may be connected to a first longitudinal end of a further neighboring, elongated conductor section, i.e. the third elongated conductor section. An opposite end, hence the second end of the third elongated conductor section may be connected to a second end of a fourth conductor section, and so on. A free end section of a last longitudinal conductor section may be connected to the second electrode or may constitute the second electrode. With a meandering conductive structure comprising a total number of three elongated and parallel oriented conductor sections, the second end of the third elongated conductor section may be connected to the second electrode or may form the second electrode.

According to a further embodiment the signal generator comprises a signal generating portion. At least the signal generating portion or the entire signal generator is made of a material having a relative permittivity εᵣ that is larger than 3, larger than 4, larger than 5, larger than 6, larger than 7, larger than 10, larger than 12 or larger than 15. With typical implementations the signal generating portion of the signal generator comprises or is made of polyamide, silicon dioxide, neoprene, natural or synthetic rubber, graphite, silicon or other materials comprising a relative permittivity εᵣ larger than 3.

With some examples the signal generator comprises a signal generating portion coated, covered or made of an elastomeric material, such as natural or synthetic rubber. Rubber comprises a comparatively large relative permittivity, larger than 5 or even larger than 6. As soon as the signal generating portion of the signal generator gets in direct vicinity of the interdigital electrode structure and as soon as the signal generating portion penetrates or traverses the electric field provided and generated by the interdigital electrode structure a measurable signal is obtained at the processor thus indicating that the at least one signal generator has passed by the at least one sensor.

According to another example the interdigital electrode structure is configured to generate a magnetic field. Here, the at least one signal generator is configured to modify the magnetic field. Here, the interdigital electrode structure comprises at least a first or primary winding and at least one second or secondary winding. When appropriately driven by a driving voltage the first or primary winding generates a spatially periodic magnetic field and the second or secondary winding is or are implemented as inductive windings, e.g. in form of a simple turn of wire. The second electrode or second windings are configured and operable to sense a magnetic field generated by the first electrode, first winding or primary windings. Any modification of the magnetic field generated by the first electrodes or windings is detectable and/or quantitatively measurable by the at least second electrodes or windings. Here, the at least one signal generator comprises a diamagnetic, paramagnetic or ferromagnetic material capable to modify the magnetic field generated or provided by the interdigital electrode structure. With this example the at least one sensor can be implemented as a so called meandering winding magnetometer (MWM).

With another example the at least one sensor is arranged at a predefined radial sensor distance D from the axis of rotation. Moreover, the at least one signal generator is arranged at a predefined radial signal generator distance d from the axis of rotation. Here, a difference between the radial sensor distance and the radial signal generator distance (D−d) is smaller than or equal to a difference between a radial extent of the at least one sensor and a radial extent of the at least one signal generator. In this way it is provided that the at least one sensor and the at least one signal generator are enabled to overlap in radial and axial direction as the first member is subject to a rotation relative to the second member with regard to the axis or rotation.

According to another example numerous sensors of the at least one sensor are distributed across one side of the second member. Alternatively or additionally, the numerous signal generators of the at least one signal generator are distributed across one side of the first member facing towards the second member. In either way, there are provided multiple signal generators and/or multiple sensors. The numerous sensors and signal generators can be equidistantly or equiangularly distributed along the circumference of the first member and the second member, respectively. For instance, if the first member is provided with four signal generators equidistantly arranged along the circumference of the first member and if the second member comprises only one sensor the angular resolution of the rotation sensing arrangement will be as small as 90°. By making use of numerous sensors regularly or irregularly distributed along the circumference of the second member the spatial resolution can be modified, e.g. increased, depending on the number of sensors and depending on the specific separation between neighboring sensors.

It is for instance conceivable, that the first number of sensors is provided on the second member and that a second number of signal generators is provided on the first member, wherein the first and the second numbers are unequal. Here, numerous regular or irregular spatial patterns are sensors on the second member in combination with a regular or irregular pattern of numerous signal generators on the first member may provide an unequivocal rotary encoder between the first member and the second member thus allowing to determine the sense of rotation as well as the degree of rotation of the first member relative to the second member.

According to another example the at least one sensor and the at least one signal generator are permanently out of mechanical contact. Hence, the at least one sensor and the at least one signal generator are arranged on the second member and on the first member, respectively in a collisionless way. Especially with the implementation of the interdigital electrode structure as an interdigital capacitor or as an MWM the passing of the at least one signal generator by the at least one sensor is detectable in a contactless way. A contactless electrical and/or magnetic measurement is of particular benefit to extend the overall lifetime of the rotation sensing arrangement because there is no friction between the first member and the second member. Moreover, neither the at least one sensor nor the at least one signal generator are subject to wear or abrasion.

In another example the planar substrate is a piezoelectric substrate. The interdigital electrode structure is arranged on the piezoelectric substrate and the arrangement of the interdigital electrode structure on the piezoelectric substrate is operable to generate an electrical signal in response to a surface acoustic wave on or through the planar substrate. In this way and by implementing the planar substrate as a piezoelectric substrate the at least one sensor can be implemented as an interdigital transducer. The interdigital transducer comprises a first and a second electrode, typically of comb-like shape. The first and second electrodes, hence the comb structures may be arranged in the fashion of a zipper, namely with the free ends of the teeth of the comb structures facing towards each other and with the free space between the teeth of one comb structure receiving the teeth of the other comb structure.

The interdigital transducer is operable to convert a mechanical vibration, e.g. a surface acoustic wave on the planar substrate into an electric signal via the piezoelectric effect. In this way, any surface acoustic waves present on the second member can be detected. Interdigital transducers are commercial available on the market and can be easily implemented in a rather miniaturized way on or in the second member.

With another example the rotation sensing arrangement comprises a strain gauge. Here, the interdigital electrode structure is part of the strain gauge attached to the second member. The interdigital electrode structure exhibits a measurable variation in electrical conductance in response to a flexible deformation. Through a mechanical connection between the strain gauge and the second member the interdigital electrode structure exhibits a measurable variation in electrical conductance in response to a flexible deformation of the second member.

Typically, the second member is subject to a flexible deformation when subject to a rotation relative to the first member. For this, the first member and the second member may be in an at least temporal mechanical engagement. For instance, the first member and the second member may be mechanically engaged by a ratchet arrangement. As the first member is rotated relative to the second member the second member is subject to a flexible deformation, e.g. a regular and repetitive flexible deformation. Since the interdigital electrode structure is attached, e.g. adhesively attached, to the second member the deformation of the second member equally transfers to a respective deformation of the interdigital electrode structure. Accordingly, the electrical resistance of the interdigital electrode structure is subject to a measurable change as the second member undergoes an elastic deformation.

The strain gauge may comprise an insulating flexible backing supporting a conductive, e.g. metallic, interdigital pattern. The insulating flexible backing and/or the conductive interdigital pattern may be attached to the second member by a suitable adhesive, such as cyanocrylate. The conductive interdigital pattern may comprise a constantan alloy.

The at least one sensor may comprise a first strain gauge and a second strain gauge, both comprising an interdigital electrode structure. Here, the first strain gauge may serve as a reference electrical resistor and the second strain gauge may be subject to elastic deformation and may hence serve as a measurement electrical resistor. The first and the second strain gauge may be mutually electrically connected by a Wheatstone bridge. Then and for measuring of mechanical strain or load present to the second member it is generally sufficient to determine a variation of the electrical resistivity of the first strain gauge relative to the second strain gauge.

One of the first strain gauge and the second strain gauge may be integrated into the printed circuit board whereas the other one of the first strain gauge and the second strain gauge may be located remote from the printed circuit board. Typically, the first strain gauge and hence the reference electrical resistor may be integrated into the printed circuit board whereas the second strain gauge is adhesively attached to an elastically deformable portion of the second member.

An orientation of the interdigital electrode structure of the strain gauge on the second member is typically in line or parallel to a predominant direction of flexible deformation of the second member as the second member rotates relative to the first member. In this way, a measurement sensitivity and hence a measurement precision can be enhanced.

According to another example the rotation sensing arrangement comprises at least one ratchet arrangement engaged with at least one of the first member and the second member. The ratchet arrangement is configured to support a rotation of the first member relative to the second member in discrete rotational steps. Typically, the ratchet arrangement comprises a first ratchet member located on the first member and further comprises a second ratchet member located on the second member. The first ratchet member faces towards the second ratchet member and vice versa. The first ratchet member typically comprises a first protrusion, e.g. in form of a tooth, facing towards the second ratchet member.

The second ratchet member typically comprises a second protrusion facing towards the first ratchet member. The second ratchet member may also comprise a tooth or a toothed structure. With some examples, the first ratchet member comprises a toothed rim with a toothed structure on an outside surface or on an inside surface. The second ratchet member may comprise a resiliently deformable ratchet member to regularly engage with the first ratchet member as the first and second ratchet members are subject to a rotation relative to each other with regard to the axis or rotation. In this way, the first member can be rotated in discrete steps relative to the second member, e.g. in a dose incrementing direction and/or in a dose decrementing direction.

The size of these discrete steps is governed by a periodicity of at least one of the first ratchet member and the second ratchet member. A resiliently deformable or resiliently biased ratchet member is either resiliently deformable in radial direction, e.g. radially inwardly or radially outwardly. Alternatively, the respective ratchet member is resiliently deformable in axial direction. In any case, the ratchet engagement between the first member and the second member provides and generates a surface acoustic wave in at least one of the first member and the second member. As the first member is rotated relative to the second member by discrete rotational steps as defined and governed by the ratchet engagement between the first and the second member an audible click sound may be generated thus indicating to the user that the first member has been rotated relative to the second member by a discrete angular distance, which may correspond to a predefined dose size, e.g. one international unit.

Generally, the second ratchet member may be equipped or provided with the at least one sensor comprising an electrical strain gauge exhibiting a measurable variation of its electrical conductance in response to a flexible deformation of the second member. The electrical strain gauge comprises the interdigital electrode structure.

With some examples the first member and the hence the first ratchet member can be integrated into the housing of the injection device or into the housing of the add-on device whereas the second member of the rotation sensing arrangement is rotatable relative to the housing. But also other examples are conceivable, wherein the first member is rotatable relative to the housing and wherein the second member of the rotation sensing arrangement is integrated into or is permanently and rigidly connected to the housing of the injection device or add-on device.

The implementation of the ratchet arrangement is of particular benefit to generate surface acoustic waves that can be detected by the at least one sensor, namely when the sensor is implemented as an interdigital transducer.

According to another aspect the disclosure further relates to an injection device for setting and expelling of a dose of a medicament. The injection device comprises a housing and a trigger to initiate and/or to control expelling of the dose. The injection device further comprises a dial member rotatable relative to the housing for setting of the dose. The injection device further comprises at least one rotation sensing arrangement as described above, wherein the first member and the second member are integrated into the injection device. Typically, the first member is rotationally locked or connected to one of the dial member and the housing and the second member is rotatable relative to the other one of the dial member and the housing. It is even conceivable, that the first member is integrated into one of the dial member and the housing and that the second member is integrated into the other one of the dial member and the housing.

Instead of the dial member or the housing the first member can be also connected to or integrated into a rotatable component of a drive mechanism of the injection device, which rotatable component is rotationally locked to the dial member. Likewise, the other one of the first member and the second member can be integrated into the housing or can be rigidly fastened or attached to a component that is immovably connected or fixed to the housing.

According to another aspect the disclosure also relates to an add-on device configured for attachment to an injection device. The add-on device comprises a body configured for attachment to the dial member of the injection device. The add-on device further comprises a housing configured for attachment to the housing of the injection device. The add-on device comprises a rotation sensing arrangement as described above, wherein the first member is rotationally locked to one of the body and the housing of the add-on device and wherein the second member is rotatable relative to the other one of the body and the housing of the add-on device.

In a further aspect the disclosure also relates to a method of detecting and/or quantitatively measuring a rotation of a first member of an injection device relative to a second member of the injection device as described above. The method comprises the steps of inducing a torque to one of the first member and the second member of the rotation sensing arrangement, typically during setting of a dose. The torque is induced to one of the first member and the second member relative to the other one of the first member and the second member, thereby moving the at least one signal generator relative to the at least one sensor of the rotation sensing arrangement.

In a further step, an electrical signal of the interdigital electrode structure of the at least one sensor is measured in response to the movement of the at least one signal generator relative to the at least one sensor. In other words, the electrical signal of the interdigital electrode structure is measured as the first member is rotated relative to the second member.

In a further step, the electric signal provided by the interdigital electrode structure is processed by the processor and the angle of rotation of the first member relative to the second member is calculated on the basis of the electrical signal.

Typically, the method is conductible by a rotation sensing arrangement as described above, which rotation sensing arrangement may be implemented in an injection device, e.g. implemented as a hand-held pen-type injector. Alternative, the method is conductible by an add-on device configured for attachment to such an injection device. The injection device may be implemented as a disposable injection device intended to become discarded in its entirety once the medicament located therein has been used up or should no longer be used. The rotation sensing arrangement is likewise implementable in a reusable device configured for multiple and long-lasting use, wherein a medicament cartridge is intended to become replaced as it is empty or as the medicament located therein should no longer be used.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The term “drug” or “medicament”, as used herein, means a pharmaceutical formulation containing at least one pharmaceutically active compound, wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound, wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis, wherein in a further embodiment the pharmaceutically active compound comprises at least one peptide for the treatment and/or prophylaxis of diabetes mellitus or complications associated with diabetes mellitus such as diabetic retinopathy, wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 Exendin-4(1-39),
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence
des Pro36 Exendin-4(1-39)-Lys6-NH2 (AVE0010),
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(02)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(02)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, K or A, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE FIGURES

In the following, numerous examples of the container and of an injection device will be described in greater detail by making reference to the drawings, in which:

FIG. 1 shows an example of an injection device,

FIG. 2 shows the injection device of FIG. 1 in an exploded perspective view,

FIG. 3 shows a block diagram of a the sensor arrangement for use with drug delivery device or injection device,

FIG. 4 is a schematic perspective illustration of an integration of the sensor arrangement in a dosing arrangement of an injection device,

FIG. 5 schematically illustrates one implementation of the sensor arrangement in a top view,

FIG. 6 shows the example of FIG. 5 in a perspective side view,

FIG. 7 schematically illustrates an interdigital electrodes structure of the sensor,

FIG. 8 schematically illustrates a cross-section of the interdigital electrode structure,

FIG. 9 schematically illustrates a further example of the sensor arrangement,

FIG. 10 illustrates another example of the sensor arrangement,

FIG. 11 schematically illustrates an implementation of the interdigital electrode structure forming a meandering winding magnetometer,

FIG. 12 is illustrative of a method of measuring a rotation using the sensor arrangement, and

FIG. 13 schematically illustrates an example of an interdigital electrode structure implemented as a strain gauge.

### DETAILED DESCRIPTION

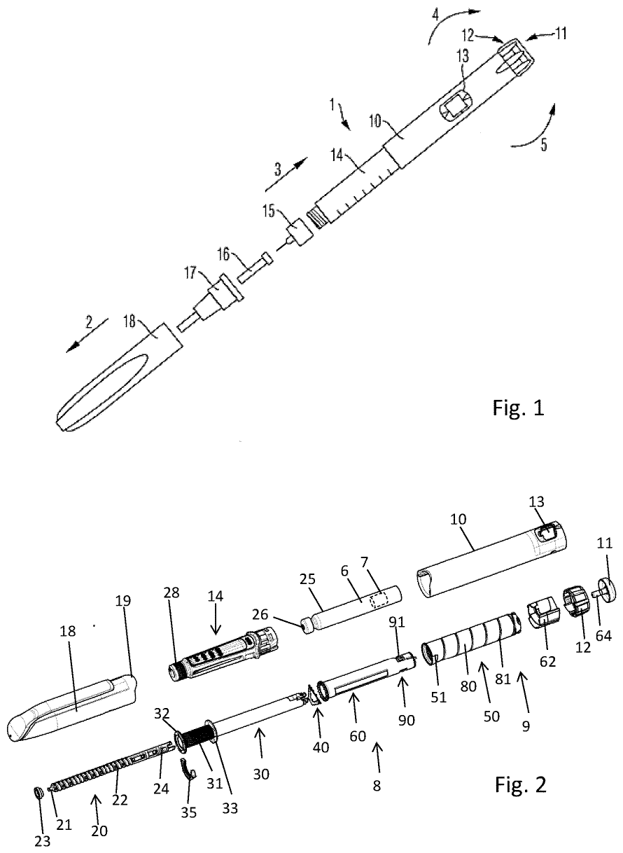

One example of an injection device 1 suitable for implementation of the rotation sensing arrangement is shown in FIGS. 1 and 2. The injection device 1 is a pre-filled disposable injection device that comprises a housing 10 to which an injection needle 15 can be affixed. The injection needle 15 is protected by an inner needle cap 16 and either an outer needle cap 17 or a protective cap 18 that is configured to enclose and to protect a distal section of the housing 10 of the injection device 1. The housing 10 may comprise and form a main housing part configured to accommodate a drive mechanism 8 as shown in FIG. 2. The injection device 1 may further comprise a distal housing component denoted as cartridge holder 14. The cartridge holder 14 may be permanently or releasably connected to the main housing 10. The cartridge holder 14 is typically configured to accommodate a cartridge 6 that is filled with a liquid medicament. The cartridge 6 comprises a cylindrically-shaped or tubular-shaped barrel 25 sealed in proximal direction 3 by means of a bung 7 located inside the barrel 25. The bung 7 is displaceable relative to the barrel 25 of the cartridge 6 in a distal direction 2 by means of a piston rod 20. A distal end of the cartridge 6 is sealed by a pierceable seal 26 configured as a septum and being pierceable by a proximally directed tipped end of the injection needle 15. The cartridge holder 14 comprises a threaded socket 28 at its distal end to threadedly engage with a correspondingly threaded portion of the injection needle 15. By attaching the injection needle 15 to the distal end of the cartridge holder 14 the seal 26 of the cartridge 6 is penetrated thereby establishing a fluid transferring access to the interior of the cartridge 6.

When the injection device 1 is configured to administer e.g. human insulin, the dosage set by a dose dial 12 at a proximal end of the injection device 1 may be displayed in so-called international units (IU, wherein 1 IU is the biological equivalent of about 45.5 μg of pure crystalline insulin ( 1/22 mg). The dose dial 12 may comprise or may form a dose dial.

As shown further in FIGS. 1 and 2, the housing 10 comprises a dosage window 13 that may be in the form of an aperture in the housing 10. The dosage window 13 permits a user to view a limited portion of a number sleeve 80 that is configured to move when the dose dial 12 is turned, to provide a visual indication of a currently set dose. The dose dial 12 is rotated on a helical path with respect to the housing 10 when turned during setting and/or dispensing or expelling of a dose.

The injection device 1 may be configured so that turning the dosage knob 12 causes a mechanical click sound to provide acoustical feedback to a user. The number sleeve 80 mechanically interacts with a piston in the insulin cartridge 6. When the needle 15 is stuck into a skin portion of a patient, and when the trigger 11 or injection button is pushed, the insulin dose displayed in display window 13 will be ejected from injection device 1. When the needle 15 of the injection device 1 remains for a certain time in the skin portion after the trigger 11 is pushed, a high percentage of the dose is actually injected into the patient's body. Ejection of an insulin dose may also cause a mechanical click sound, which is however different from the sounds produced when using the dose dial 12.

In this embodiment, during delivery of the insulin dose, the dose dial 12 is turned to its initial position in an axial movement, that is to say without rotation, while the number sleeve 80 is rotated to return to its initial position, e.g. to display a dose of zero units.

The injection device 1 may be used for several injection processes until either the cartridge 6 is empty or the expiration date of the medicament in the injection device 1 (e.g. 28 days after the first use) is reached.

Furthermore, before using injection device 1 for the first time, it may be necessary to perform a so-called “prime shot” to remove air from the cartridge 6 and the needle 15, for instance by selecting two units of the medicament and pressing trigger 11 while holding the injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user.

An example of the drive mechanism 8 is illustrated in more detail in FIG. 2. It comprises numerous mechanically interacting components. A flange like support of the housing 10 comprises a threaded axial through opening threadedly engaged with a first thread or distal thread 22 of the piston rod 20. The distal end of the piston rod 20 comprises a bearing 21 on which a pressure foot 23 is free to rotate with the longitudinal axis of the piston rod 20 as an axis of rotation. The pressure foot 23 is configured to axially abut against a proximally facing thrust receiving face of the bung 7 of the cartridge 6. During a dispensing action the piston rod 20 rotates relative to the housing 10 thereby experiencing a distally directed advancing motion relative to the housing 10 and hence relative to the barrel 25 of the cartridge 6. As a consequence, the bung 7 of the cartridge 6 is displaced in distal direction 2 by a well-defined distance due to the threaded engagement of the piston rod 20 with the housing 10.

The piston rod 20 is further provided with a second thread 24 at its proximal end. The distal thread 22 and the proximal thread 24 are oppositely handed.

There is further provided a drive sleeve 30 having a hollow interior to receive the piston rod 20. The drive sleeve 30 comprises an inner thread threadedly engaged with the proximal thread 24 of the piston rod 20. Moreover, the drive sleeve 30 comprises an outer threaded section 31 at its distal end. The threaded section 31 is axially confined between a distal flange portion 32 and another flange portion 33 located at a predefined axial distance from the distal flange portion 32. Between the two flange portions 32, 33 there is provided a last dose limiter 35 in form of a semi-circular nut having an internal thread mating the threaded section 31 of the drive sleeve 30.

The last dose limiter 35 further comprises a radial recess or protrusion at its outer circumference to engage with a complementary-shaped recess or protrusion at an inside of the sidewall of the housing 10. In this way the last dose limiter 35 is splined to the housing 10. A rotation of the drive sleeve 30 in a dose incrementing direction 4 or clockwise direction during consecutive dose setting procedures leads to an accumulative axial displacement of the last dose limiter 35 relative to the drive sleeve 30. There is further provided an annular spring 40 that is in axial abutment with a proximally facing surface of the flange portion 33. Moreover, there is provided a tubular-shaped clutch 60. At a first end the clutch 60 is provided with a series of circumferentially directed saw teeth. Towards a second opposite end of the clutch 60 there is located a radially inwardly directed flange.

Furthermore, there is provided a dose dial sleeve also denoted as number sleeve 80. The number sleeve 80 is provided outside of the spring 40 and the clutch 60 and is located radially inward of the housing 10. A helical groove 81 is provided about an outer surface of the number sleeve 80. The housing 10 is provided with the dosage window 13 through which a part of the outer surface of the number 80 can be seen. The housing 10 is further provided with a helical rib at an inside sidewall portion of an insert piece 62, which helical rib is to be seated in the helical groove 81 of the number sleeve 80. The tubular shaped insert piece 62 is inserted into the proximal end of the housing 10. It is rotationally and axially fixed to the housing 10. There are provided first and second stops on the housing 10 to limit a dose setting procedure during which the number sleeve 80 is rotated in a helical motion relative to the housing 10. As will be explained below in greater detail, at least one of the stops is provided by a preselector stop feature 71 provided on a preselector 70.

The dose dial 12 in form of a dose dial grip is disposed about an outer surface of the proximal end of the number sleeve 80. An outer diameter of the dose dial 12 typically corresponds to and matches with the outer diameter of the housing 10. The dose dial 12 is secured to the number 80 to prevent relative movement there between. The dose dial 12 is provided with a central opening.

The trigger 11, also denoted as dose button is substantially T-shaped. It is provided at a proximal end of the injection device 10. A stem 64 of the trigger 11 extends through the opening in the dose dial 12, through an inner diameter of extensions of the drive sleeve 30 and into a receiving recess at the proximal end of the piston rod 20. The stem 64 is retained for limited axial movement in the drive sleeve 30 and against rotation with respect thereto. A head of the trigger 11 is generally circular. The trigger side wall or skirt extends from a periphery of the head and is further adapted to be seated in a proximally accessible annular recess of the dose dial 12.

To dial a dose a user rotates the dose dial 12. With the spring 40 also acting as a clicker and the clutch 60 engaged, the drive sleeve 30, the spring or clicker 40, the clutch 60 and the number sleeve 80 rotate with the dose dial 12. Audible and tactile feedback of the dose being dialed is provided by the spring 40 and by the clutch 60. Torque is transmitted through saw teeth between the spring 40 and the clutch 60. The helical groove 81 on the number sleeve 80 and a helical groove in the drive sleeve 30 have the same lead. This allows the number sleeve 80 to extend from the housing 10 and the drive sleeve 30 to climb the piston rod 20 at the same rate. At a limit of travel a radial stop on the number sleeve 80 engages either with a first stop or a second stop provided on the housing 10 to prevent further movement in a first sense of rotation, e.g. in a dose incrementing direction 4. Rotation of the piston rod 20 is prevented due to the opposing directions of the overall and driven threads on the piston rod 20.

The last dose limiter 35 keyed to the housing 10 is advanced along the threaded section 31 by the rotation of the drive sleeve 30. When a final dose dispensed position is reached, a radial stop formed on a surface of the last dose limiter 35 abuts a radial stop on the flange portion 33 of the drive sleeve 30, preventing both, the last dose limiter 35 and the drive sleeve 30 from rotating further.

Should a user inadvertently dial beyond the desired dosage, the injection device 1, configured as a pen-injector allows the dosage to be dialed down without dispense of the medicament from the cartridge 6. For this the dose dial 12 is simply counter-rotated. This causes the system to act in reverse. A flexible arm of the spring or clicker 40 then acts as a ratchet preventing the spring 40 from rotating. The torque transmitted through the clutch 60 causes the saw teeth to ride over one another to create the clicks corresponding to dialed dose reduction. Typically, the saw teeth are so disposed that a circumferential extent of each saw tooth corresponds to a unit dose. Here, the clutch may serve as a ratchet mechanism.

As an alternative or in addition the ratchet mechanism 90 may comprise at least one ratchet feature 91, such as a flexible arm on the sidewall of the tubular-shaped clutch 60. The at least one ratchet feature 91 may comprise a radially outwardly extending protrusion e.g. on a free end of the flexible arm. The protrusion is configured to engage with a correspondingly shaped counter ratchet structure on an inside of the number sleeve 80. The inside of the number sleeve 80 may comprise longitudinally shaped grooves or protrusions featuring a saw-tooth profile. During dialing or setting of a dose the ratchet mechanism 90 allows and supports a rotation of the number sleeve 80 relative to the clutch 60 along a second sense of rotation 5, which rotation is accompanied by a regular clicking of the flexible arm of the clutch 60. An angular momentum applied to the number sleeve 80 along the first sense of rotation for is unalterably transferred to the clutch 60. Here, the mutually corresponding ratchet features of the ratchet mechanism 90 provide a torque transmission from the number sleeve 80 to the clutch 60.

When the desired dose has been dialed the user may simply dispense the set dose by depressing the trigger 11. This displaces the clutch 60 axially with respect to the number sleeve 80 causing dog teeth thereof to disengage. However, the clutch 60 remains keyed in rotation to the drive sleeve 30. The number sleeve 80 and the dose dial 12 are now free to rotate in accordance with the helical groove 81.

The axial movement deforms the flexible arm of the spring 40 to ensure the saw teeth cannot be overhauled during dispense. This prevents the drive sleeve 30 from rotating with respect to the housing 10 though it is still free to move axially with respect thereto. The deformation is subsequently used to urge the spring 40 and the clutch 60 back along the drive sleeve 30 to restore the connection between the clutch 60 and the number sleeve 80 when the distally directed dispensing pressure is removed from the trigger 11.

The longitudinal axial movement of the drive sleeve 30 causes the piston rod 20 to rotate through the through opening of the support of the housing 10, thereby to advance the bung 7 in the cartridge 6. Once the dialed dose has been dispensed, the number sleeve 80 is prevented from further rotation by contact of at least one stop extending from the dose dial 12 with at least one corresponding stop of the housing 10. A zero dose position may be determined by the abutment of one of axially extending edges or stops of the number sleeve 80 with at least one or several corresponding stops of the housing 10.

The expelling mechanism or drive mechanism 8 as described above is only exemplary for one of a plurality of differently configured drive mechanisms that are generally implementable in a disposable pen-injector. The drive mechanism as described above is explained in more detail e.g. in WO2004/078239A1, WO 2004/078240A1 or WO 2004/078241A1 the entirety of which being incorporated herein by reference.

The dose setting mechanism 9 as illustrated in FIG. 2 comprises at least the dose dial 12 and the number sleeve 80. As the dose dial 12 is rotated during and for setting of a dose the number sleeve 80 starts to rotate relative to the housing along a helical path as defined by the threaded engagement of its outer thread or helical groove 81 with a correspondingly shaped threaded section at the inside surface of the housing.

During dose setting and when the drive mechanism 8 or the dose setting mechanism 9 is in the dose setting mode the drive sleeve 30 rotates in unison with the dose dial 12 and with the number sleeve 80. The drive sleeve 30 is threadedly engaged with the piston rod 20, which during dose setting is stationary with regard to the housing 10. Accordingly, the drive sleeve 30 is subject to a screwing or helical motion during dose setting. The drive sleeve 30 starts to travel in proximal direction as the dose dial is rotated in a first sense or rotation or in a dose incrementing direction 4, e.g. in a clockwise direction. For adjusting of or correcting a size of a dose the dose dial 12 is rotatable in an opposite second sense of rotation, hence in a dose decrementing direction 5, e.g. counterclockwise.

Numerous examples of a rotation sensing arrangement 200 for use with an injection device 1 or for use with an add-on device 100 attachable to such an injection device 1 are illustrated in FIGS. 4-11. The rotation sensing arrangement 200 is configured for detecting and/or measuring a rotational motion of a first member 201 relative to a second member 202 of an injection device 1 or of an add-on device 100 configured for mechanical attachment to such an injection device 1 as for instance illustrated in FIG. 1 or 2.

The rotation sensing arrangement 200 as shown in FIGS. 4-6 comprises a first member 201 and a second member 202. The first member 201 is rotatable relative to the second member 202 with regards to an axis of rotation 203. Typically, the first member 201 and the second member 202 are arranged coaxial with regards to the axis of rotation 203. With some examples, the first member 201 and the second member 202 are arranged axially adjacent with regard to the axis of rotation 203. The first member 201 and the second member 202 may be directly mechanically engaged. With other examples, the first member 201 and the second member 202 are mechanically disengaged from each other. Here, the first member 201 and the second member 202 may be separately arranged or rotationally supported in or at a housing 10 of the injection device 4 or in or at a respective housing of a separate add-on device.

At least one of the first member 201 and the second member 202 is typically rotationally supported in or on the housing 10 of the injection device 1. With some examples, both, the first member 201 and the second member 202 can be rotationally supported on or with regard to the housing 10. Typically, and depending on the specific implementation or integration of the rotation sensing arrangement 200 in the injection device 1 one of the first member 201 and the second member 202 is rotationally locked to the housing 10 whereas the other one of the first member 201 and the second member 202 is rotationally movable relative to the housing 10. Typically, one of the first member 201 and the second member 202 is rotatable relative to the housing 10 with regard to the axis of rotation 203.

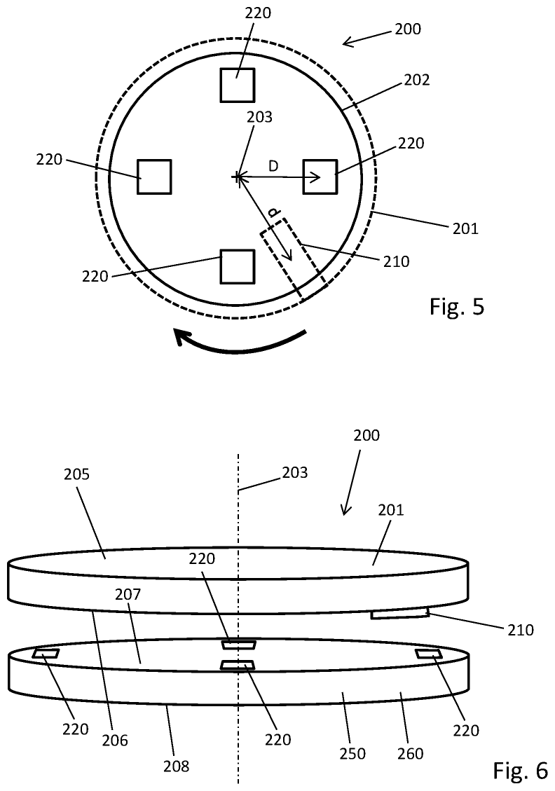

As it is illustrated in more detail in FIGS. 5 and 6 the first member 201 comprises at least one signal generator 210. The second member 202 comprises at least one sensor 220. With some examples, at least one of the first member 201 and the second member 202 comprises a disc or a disc-like shape with a first and a second axial planar surface. For instance and as illustrated in FIG. 6, the first member 201 comprises an upper, e.g. a proximal axial surface 205 and a lower, e.g. distal axial surface 206. Likewise, the second member 202 aligned coaxial to the first member 201 but positioned and arranged at a predefined axial distance from the first member 201 comprises an upper or proximal surface 207 and an oppositely located lower or distal surface 208.

As indicated in FIG. 6 the distal surface 206 of the first member 201 faces towards the second member 202. Accordingly, the proximal surface 207 of the second member 202 faces towards the first member 201. The distal surface 206 of the first member 201 faces towards the proximal surface 207 of the second member 202.

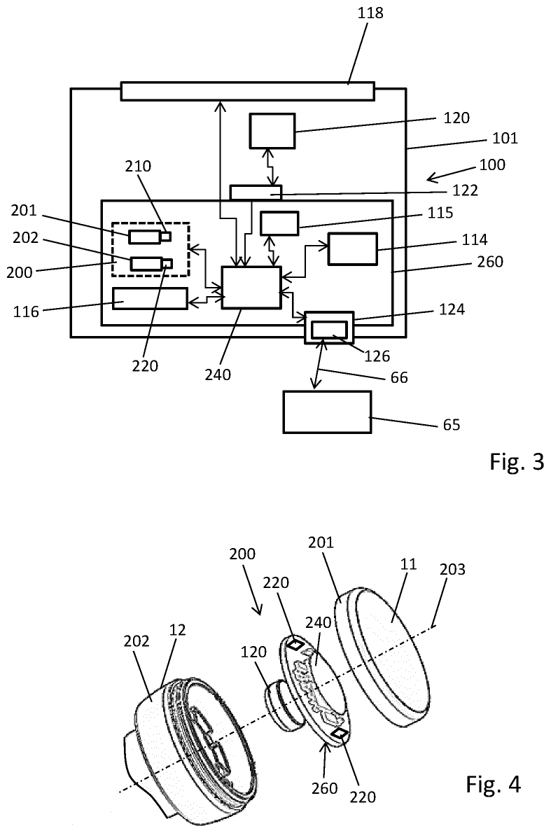

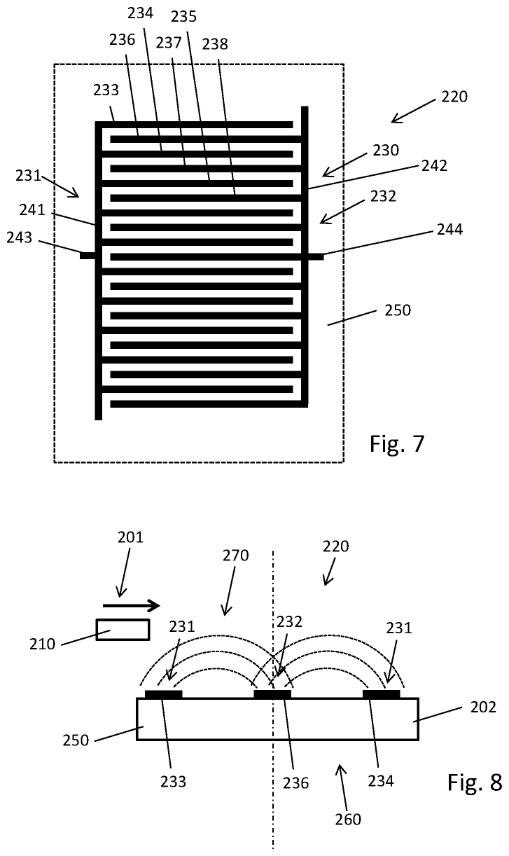

As shown in FIGS. 5 and 6, there are provided four individual sensors 220 on the second member 202. Each one of these sensors 220 comprises an interdigital electrode structure 230 or interdigital electronic structure as shown in more detail in FIGS. 7 and 8. Each one of the sensors 220 is connected to the processor 240 as illustrated in FIG. 3 or 4. The processor 240 is connected to each one of the sensors 220 in a signal transferring way. Typically and as the first member 201 is subject to a rotation relative to the second member 202 and when the signal generator 210 passes by one of the sensors 220 the respective sensor 220 is configured and operable to generate an electric signal that is processable or detectable by the processor 240. With the example of FIGS. 5 and 6 and when arranging four equidistantly spaced sensors 220 on the second member 202 and when having only one signal generator 210 on the first member 201 a rotation of the first member 201 relative to the second member 202 by at least 90° can be detected and/or precisely measured.

The presently illustrated arrangement of the number of sensors 220 on the second member 202 and the arrangement of the signal generator 210 on the first member 201 is only one of a plurality of examples and is just provided for illustration purpose. The planar spatial extension of the sensors 220 may be as small as a few millimeters in each direction. So with typical implementations numerous sensors 220, e.g. up to 8 sensors, up to 12 sensors, up to 24 sensors or even more than 36 sensors can be arranged on the annular circumference of the second member 202. In this way, the angular or spatial resolution of the rotation sensing arrangement 200 can be increased.

The rotation sensing arrangement 200 typically comprises a planar substrate 250. The planar substrate 250 may coincide with or may be provided by a printed circuit board 260 as illustrated in FIG. 4. On the printed circuit board 260 there may be provided the processor 240 together with the at least one sensor 220. Typically, the at least one sensor 220, in particular the interdigital electrode structure 230 of the respective sensors 220 can be directly printed or coated on the planar substrate 250 and/or on the printed circuit board 260. As indicated in FIG. 4, the printed circuit board 260 may be further provided with a power source 120. The power source 120 may be located on one side of the printed circuit board 260. On the same side or on the opposite side of the printed circuit board 260 there may be provided the processor 240 and/or the at least one sensor 220.

The printed circuit board 260 may be fastened to the second member 202. The second member 202 may coincide with the dial member 12 of the injection device 1. Hence, the planar substrate 250 and/or the printed circuit board 260 with the processor 240 and the at least one sensor 220 arranged thereon can be rigidly fastened to the dial member 12, hence to the second member 202. The first member 201 may be implemented as a depressible trigger 11. The first member 201 and hence the trigger 11 may be rotationally locked to the housing 10 during and/or for setting of a dose, during which the dose dial 12 and hence the second member 202 is subject to a rotation relative to the housing 10.

The implementation of the rotation sensing arrangement 200 as indicated in FIG. 4 is only one of numerous possibilities. With other examples, the first member 201 might be rotatable during and/or for setting of a dose whereas the second member 202 is rotationally locked to the housing during and/or for setting of a dose. For instance, one of the first member 201 and the second member 202 can be connected to or integrated into the number sleeve 80 whereas the other one of the first member 201 and the second member 202 is connected to or integrated into the clutch 60 of the injection device 1.

With the example of FIGS. 4-6 both, the first member 201 and the second member 202 are of circular, annular or disc-like shape. For the general working principle of the rotation sensing arrangement 200 it is sufficient, when only one or at least one of the first member 201 an the second member 202 comprises a disc-like, circular or annular structure whereas the other one of the first member 201 can be arbitrary shape or structure. Typically, that component of the first member 201 and the second member 202 that is provided with numerous sensors 220 or numerous signal generators 210 comprises a circular, annular and/or disc-like structure so as to provide a suitable rotary encoding configured to detect and to measure a degree of rotation of the first member 201 relative to the second member 202.

In the presently illustrated example, wherein the first member 201 and the second member 202 are coaxially aligned with regard to the axis of rotation 203 and wherein the first member 201 and the second member 202 are arranged at an axial distance from each other it is of particular benefit, when the at least one sensor 220 is arranged at a predefined radial sensor distance D from the axis of rotation 203. The at least one signal generator 210 is arranged at a predefined radial signal generator distance d from the axis of rotation. Here, the radial sensor distance D and the radial signal generator distance d is measured from a radial center point of the at least one sensor 220 and of the at least one signal generator 210, respectively. With the presently illustrated example the difference between the radial sensor distance D and the radial signal generator distance d is smaller than or equal to a difference between a radial extent of the at least one sensor and a radial extent of the at least one signal generator. In this way it is provided and/or guaranteed that there is a radial and/or axial overlap between the at least one signal generator 210 and the at least one sensor 220 as the first member 201 is subject to a rotation relative to the second member 202.

Typically, the first member 201 and the second member 202 are arranged in a mutually contactless way. Hence, there is no direct mechanical engagement between the first member 201 and the second member 202. However, at least one of the first member 201 and the second member 202 may be mechanically engaged with other components of the injection device 1 or of an add-on device 100, typically with the housing 10 of the injection device 1.

With other examples, the first member 201 and the second member 202 can be arranged at the same axial position with regard to the axis of rotation. Here, the first member 201 and the second member 202 can be arranged in a nested or interleaved configuration. As an example, one of the first member 201 and the second member 202 comprises a tubular or annular-shaped hollow structure and the other one of the first member 201 and the second member 202 is arranged radially therein. For instance, the first member 201 is located radially inside the second member 202. Then, at least one of the signal generator 201 and the at least one sensor 220 is located on an outside surface of the first member 201 whereas the other one of the at least one signal generator 210 and the at least one sensor 220 is located on an inside surface of the second member 202.

One example of the at least one sensor 220 is illustrated in FIGS. 7 and 8. The sensor 220 comprises an interdigital electrode structure 230 on a planar substrate 250. The interdigital electrode structure 230 is either coated or printed on a surface of the planar substrate 250. The interdigital electrode structure 230 comprises a first electrode 231 and a second electrode 232.

The first electrode 231 is electrically insulated from the second electrode 232. The first electrode 231 comprises a digit-like or finger-like periodic pattern of parallel in-plane electrode portions 233, 234, 235. These electrode portions 233, 234, 235 extend parallel to each other. The longitudinal ends of the electrode portions 233, 234, 235 flush in a direction perpendicular to the elongation of the electrode portions 233, 234, 235.

The electrode portions 233, 234, 235 are interconnected at one longitudinal end. The opposite longitudinal end of the electrode portions 233, 234, 235 is a free end. The second electrode 232 can be shaped symmetric or identical to the shape of the first electrode 231. Also the second electrode 232 comprises a digit-like or finger-like periodic pattern of parallel in-plane electrode portions 236, 237, 238. The electrode portions 236, 237, 238 are arranged parallel. They may be of equal length. The longitudinal free end of the electrode portions 236, 237, 238 flushes in a direction perpendicular to the elongation of the electrode portions 236, 237, 238. The opposite longitudinal end of the electrode portions 236, 237, 238 are electrically interconnected by a longitudinally extending connecting portion 242.

Similarly, also the electrode portions 233, 234, 235 of the first electrode 231 are interconnected by a connecting portion 241. The electrode portions 233, 234, 235 of the first electrode 231 are separated with respect to each other along the elongation of the connecting portion 241. The longitudinal end of the electrode portions 233, 234, 235 that are connected or integrally formed with the connecting portion 241 faces away from the second electrode and in particular faces away from the connecting portion 242 of the second electrode 232. The oppositely located free end of the electrode portions 233, 234, 235, i.e. that end that faces away from the connecting portion 241 faces towards the second electrode 232 and hence towards the connecting portion 242 of the second electrode 232.

In particular, the electrode portions 233, 234, 235 of the first electrode 231 extend parallel to the electrode portions 236, 237, 238 of the second electrode 232. Moreover, the electrode portions 233, 234, 235 are located in intermediate free spaces between the electrode portions 236, 237, 238; and vice versa. Typically, the electrode portions 233, 234, 235 of the first electrode are equidistantly separated along the first connecting portion 241. Accordingly also the electrode portions 236, 237, 238 of the second electrode 232 are equidistantly separated along the second connecting portion 242.

In this way, a regular, periodic pattern of electrode portions 233, 236, 234, 237, 235, 238 is provided. This interdigital electrode structure 230 comprises numerous microstrips or combs and/or forms a grating along the elongation of the connecting portions 241, 242 of the first electrode 231 and the second electrode 232, respectively.

As further illustrated in FIG. 7 the first electrode 231 comprises a first connector 243 connected to or integrally formed with the connecting portion 241 but facing away from the numerous electrode portions 233, 234, 235. In the same way also the second electrode 232 comprises a second connector 244 connected to or integrally formed with the connecting portion 242 and facing away from the numerous electrode portions 236, 237, 238. The connectors 243, 244 are individually and separately connected to the processor 240 for signal generation, signal detection and/or signal processing.

In FIG. 8 a cross-section through the periodic pattern of the interdigital electrode structure 230 is illustrated. Here, the cross-section illustrates and electric field 270 generated by the interdigital electrode structure 230. The cross-section shows a cross-section through an alternating pattern of electrode portions 233, 236, 234 of the first electrode 231 and the second electrode 232. As the first and the second electrodes 231, 232 are arranged on an electrically insulating substrate 250 there is formed an electric field 270, e.g. in the form of a fringing electric field between electrode portions 233, 236, 234 of the first and second electrodes 231, 232 respectively.

Typically, the first and the second electrodes 231, 232 are driven at opposite polarities. They may be driven with a DC voltage or with an AC voltage. The first and second electrodes 231, 232 form an electric capacitance and hence a planar capacitor. The first and the second electrodes 231, 232 may form or constitute a so-called interdigital capacitor. In this way, an interdigital dielectrometry sensor is provided and supports a direct measurement of dielectric properties of insulating and semi-insulating materials from one side. The penetration depth and/or the range of the fringing quasi-static electric field 270 above the surface of the planar substrate 250 is proportional to the spacing between the center lines of the alternating electrode portions 233, 236, 234 of the first and the second electrodes 231, 232 respectively.

Now, if the signal generator 210 is moving through the electric field 270 a respective change in capacitance of the interdigital electrode structure 230 can be detected and measured. For this, it is not necessary that the signal generator 210 gets in mechanical contact with any of the first electrode 231 or second electrode 232. The interdigital electrode structure 230 and the signal generator 210 are typically arranged in a contactless manner. In order to have a good signal-to-noise ratio it is of particular benefit, when the signal generator comprises a relatively permittivity εᵣ larger than 3, larger than 4, larger than 5, larger than 6, larger than 7, larger than 10, larger than 12 or larger than 15. With typical implementations the signal generator 210 comprises an elastomeric material, such as natural or synthetic rubber exhibiting a relative permittivity larger than 5, larger than 6 or larger than or equal to 7.

Generally, the measuring principle of the at least one sensor 220 is not limited to an interdigital capacitor. With other implementations of the interdigital electrode structure 230 the at least one sensor 320 can be implemented as a magnetic sensor. It may be implemented as a meandering winding magnetometer as illustrated in FIG. 11. Here, the interdigital electrode structure 330 comprises a first electrode 331 implemented as a meandering electric wire or winding on the planar substrate 250. The first electrode 331 forms a primary electrode or primary winding for creating a spatially periodic magnetic field 280 when driven by an electric current.

There is further provided a second electrode 332 forming a secondary meandering winding on the planar substrate 250. The second electrode 332 is typically implemented as an inductive winding configured or operable to detect variations of the magnetic field 280 generated by the primary winding 331. Typically and when taking a measurement a time varying current is applied to the first electrode 331 or to the primary winding, which produces a time varying magnetic field. When a conductive material, such as the signal generator 210 is in close vicinity to the interdigital electrode structure 330 this has an influence on the magnetic field 280 induced in the second electrode 332, i.e. in the secondary winding.

This changing magnetic field produces a measurable signal that can be detected by the processor 240 connected to the first and second electrodes 331, 332, respectively. With some implementations and as illustrated in more detail in FIG. 11 there may be provided two secondary electrodes 332, 333. Also here, the interdigital pattern is provided and/or formed by the insulating substrate 250 and numerous windings, 331, 332, 333 as a special case of respective first, second and third electrodes 331, 332, 333. When electric current passes through the primary winding 331, it induces eddy currents in the secondary windings 332, 333. The secondary winding voltage is given by the time rate of change of magnetic flux passing through the respective winding from the current in the primary winding 331. At low frequencies, the induced voltage can become very small. To overcome such a low-frequency limitation, one can replace the secondary winding with a magnetoresistive sensor that can operate at very low frequencies, down to DC.

When the sensor 320 is implemented as a magnetic sensor, e.g. as a meandering winding magnetometer it is of particular benefit when the signal generator 210 is operable to induce a measurable modification of the magnetic field generated by the first electrode 331, hence by the primary winding.

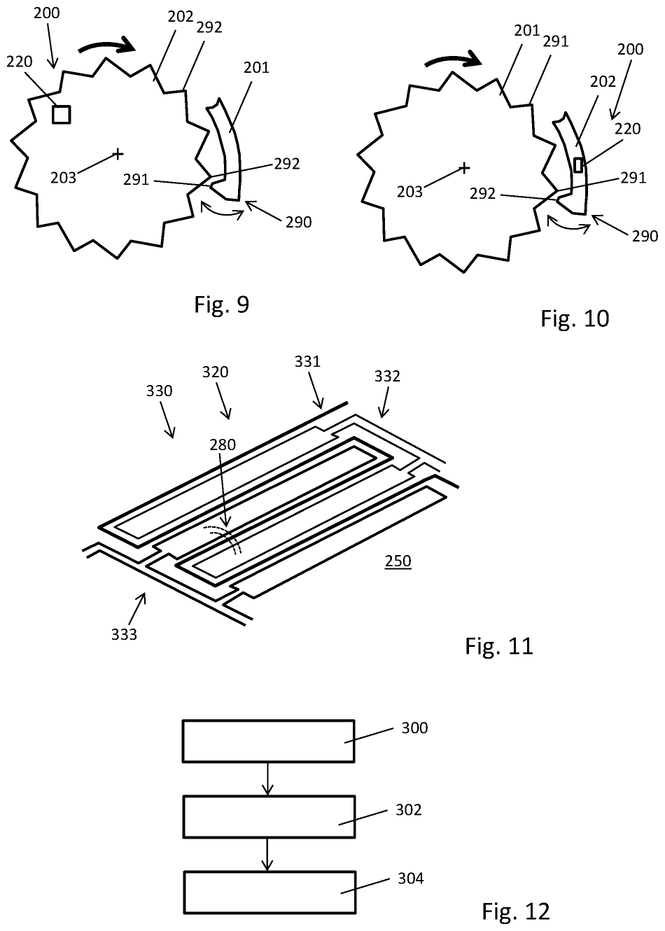

In FIGS. 9 and 10 two further implementations of a rotation sensing arrangement 200 are illustrated. Here, the first member 201 and the second member 202 are mechanically engaged, e.g. by a ratchet arrangement 290. For this, the first member 201 comprises a first ratchet member 291 configured to mechanically engage with a second ratchet member 292 of the second member 202. In this way and as the first member 201 is subject to a rotation relative to the second member 202 with regard to the axis of rotation 203 a surface acoustic wave is generated across at least one of the first member 201 and the second member 202. Here, the sensor 220 is configured to detect the presence or propagation of such a surface acoustic wave.

For detecting a surface acoustic wave and hence for detecting a mechanical excitation of the second member 202 the second member 202 or at least a portion thereof in the region of the at least one sensor 220 comprises a piezoelectric substrate 250. Here, the at least one sensor 220 is implemented as an interdigital transducer operable to generate an electric signal in response to a surface acoustic wave propagating across the surface of the second member 220. In the example of FIG. 9, it is the second member 202 that is rotatable relative to a first member 201. The second member 202 comprises a ratchet member 292 with a toothed structure on an outer or inner circumference. In the example of FIG. 9, a sidewall or an outer rim of the second member 202 comprises a regular structure of radially outwardly protruding teeth configured to engage with the first member 201, in particular with a protrusion of the first ratchet member 291. Here, the first member 201 is elastically deformable. It may be radially biased inwardly so as to engage with the radial outer toothed surface of the second member 202. There may be provided numerous first members 201 that are arranged e.g. diametrically opposite to each other with regard to the axis of rotation 203.

As an alternative to the illustrated example of FIG. 9 the second ratchet member 292 can also be implemented as an inward facing surface and the first ratchet member 291 can be located radially inwardly from the second ratchet member 292. Then, the first ratchet member 291 is biased radially outwardly so as to regularly engage with the toothed structure of the second ratchet member 292 as the second member 202 is subject to a rotation relative to the first member 201. With either implementation one of the first member 201 and the second member 202 is typically non-rotationally engaged with the housing 10 of the injection device 1 whereas the other one of the first member 201 and the second member 202 is rotationally supported relative to the housing.

In the example of FIG. 9 the at least one sensor 220 is located on the disc-shaped second member 202 whereas the first member 201 is flexibly deformable against an inherent restoring force. For instance, the first member 201 comprises an arc-shaped flexible structure. As the ratchet member 292 engages or disengages with the first ratchet member 291 a surface acoustic wave travels across the second member 202. Typically, the ratchet arrangement 290 produces an audible click sound every time the second member 202 is rotated by a discrete angular distance relative to the first member 201.

In the further example as illustrated in FIG. 10 a similar ratchet engagement 290 between the first member 201 and the second member 202 is implemented but here and compared to the example of FIG. 9 the roles of the first member 201 and the second member 202 have been swapped. The first member 201 comprises a disc-like circular or annular structure with the first ratchet member 291 on an outer or inner annular surface. The second member 202 comprises a radially protruding ratchet member 292 configured to regularly engage with the teeth of the first ratchet member 291 as the first member 201 is subject to a rotation relative to the second member 202. Here, it is the second member 202 that is elastically deformable. The second member 202 may be rotationally locked to the housing 10. Hence, it may be immobile with regard to the housing 10. As the first member 201 is rotated relative to the second member 202 with regard to the axis of rotation 203 the second member 202 is subject to an elastic deformation accompanied by the generation of a surface acoustic wave. Here, the at least one sensor 220 located on the elastically deformable second member 202 is configured or operable to detect a surface acoustic wave by the interdigital transducer provided and hence by the interdigital electrode structure 230 of the at least one sensor 220.

The examples of FIGS. 9 and 10 can be easily implemented in existing injection devices 1 because such mechanically implemented injection devices typically comprise at least one ratchet engagement 290 as schematically illustrated in FIGS. 9 and 10. In the illustrated examples of FIGS. 9 and 10 one of the first member 201 and the second member 202 is elastically deformable in radial direction.

It should be noted that the present disclosure is not limited to such radially deformable mechanical structures. Rather, the principle of rotation sensing can be likewise implemented with an axially elastically deformable first member 201 or second member 202. With the sensor 220 implemented as an interdigital transducer it is generally sufficient, when at least one of the first member 201 and the second member 202 is provided with only a single sensor 220. The at least one sensor 220 arranged on or integrated in the second member 202 can be arranged on a rotating second member or on a rotationally locked and hence non-rotating second member 202. The implementation as an interdigital transducer provides the benefit that both, the rotating member and the non-rotating member are equally subject to a surface acoustic wave as the first member and the second member 201, 202 are subject to a relative rotation accompanied by the mechanical engagement of first and second ratchet members 291, 292.

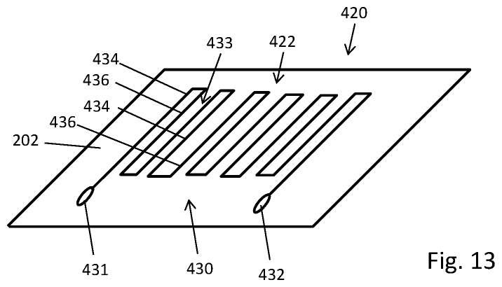

In FIG. 13, a further example of a sensor 420 is illustrated. The sensor 420 also comprises an interdigital electrode structure 430. Here, the interdigital electrode structure 430 is part of a strain gauge 422 attached to the second member 202 as illustrated for instance in FIG. 10. Hence, the sensor 420 and/or the strain gauge 422 may replace the sensor 220 as illustrated in FIG. 10. The interdigital electrode structure 430 exhibits a measurable variation in electrical conductance in response to a flexible deformation of the second member 202.

The interdigital electrode structure 430 comprises a first electrode 431 and a second electrode 432 mutually electrically connected by meandering conductive structure 433. The meandering conductive structure 433 comprises numerous elongated conductor sections 434, 436 extending parallel to each other. The numerous elongated conductor section 434, 436 are electrically connected in line. As the interdigital electrode structure 430 is subject to a variation in length, in particular along the elongation of the elongated conductor sections 434, 436, the electrical resistance between the first electrode 431 and the second electrode 432 is subject to a measurable variation. This measurable variation is detectable and/or quantitatively measurable by the processor 240 connected to the sensor 420.

Typically, the interdigital electrode structure 430 is oriented and/or arranged on the second member 202 in such a way, that the elongated conductor sections 434, 436 substantially align or extend substantially parallel to a predominant direction of elastic deformation of the second member 202. In this way, the sensitivity and/or the measurement position of the sensor 420 can be increased to a maximum.

As a consequence and as the first member 201 is subject to a rotation relative to the second member 202 the second member 202 is subject to a regular flexible deformation. This flexible deformation is detectable by the interdigital electrode structure 430 of the strain gauge 422.

In FIG. 12 the method of detecting and/or quantitatively measuring a rotation of a first member 201 relative to a second member 202 is schematically illustrated. In a first step 300 a torque is induced to one of the first member 201 relative to the second member 202 thus leading to a respective rotation of the first member 201 relative to the second member 202 with regard to the axis of rotation 203. Accordingly and since the signal generator 210 is arranged or attached to the first member and as the at least one sensor 220 is arranged on or attached to the second member 202 the at least one signal generator 210 is subject to a movement relative to the at least one sensor 220.

In a further step 302 an electric signal is generated and provided by the interdigital electrode structure 230, 330 of the at least one sensor 220, 320 in response to the movement of the at least one signal generator 210 relative to the at least one sensor 220, 320. As a consequence and in a further step 304 the electrical signal provided and generated by the processor 240 electrically connected to the at least one sensor 220 and hence to the interdigital electrode structure 230 of the at least one sensor 220 is processed. The processor 240 is configured and operable to calculate an angle of rotation of the first member 201 relative to the second member 202 on the basis of the electrical signal obtained from the at least one sensor 220, 320. Hence, in step 304, occurrence of a rotational movement and/or a degree of rotation of the first member 201 relative to the second member 202 is detected and/or determined.

FIG. 3 is a block diagram of an implementation of the rotation sensing arrangement 200 in an add-on device 100. Here, the rotation sensing arrangement 200 may be integrated into the add-on device 100. At least some components of the add-on device 100 are shared by the rotation sensing arrangement 200 and the add-on device 100. In a similar way the rotation sensing arrangement 200 can be also integrated into an injection device 1.

The add-on device 100 may comprise a data collection device. The add-on device 100 comprises the 240 of the including one or more processors, such as a microprocessor, a Digital Signal Processor (DSP), Application Specific Integrated Circuit (ASIC), Field Programmable Gate Array (FPGA) or the like, together with a memory 114. The memory may 114 include a program memory and main memory, which can store software for execution by the processor 240 and data generated during use of the add-on device 100 such as counted pulses, derived dose size, time stamp, etc. A switch 122 connects a power source 120 to the electronic components of the device 100, including the rotation sensing arrangement 200. A display 118 may or may not be present. The rotation sensing arrangement 200 is coupled to a first member 201 and a second member 202 as described above. It comprises at least one sensor 220 connected or attached to the second member 202 and further comprises at least one signal generator 210 connected or attached to the first member 201.

With the present implementation of the rotation sensing arrangement 200 in an add-on device 100 one of the first member 201 and the second member 202 can be connected or fastened to the housing 101 of the add-on device 100 and the other one of the first member 201 and the second member 202 is for instance connectable or fastenable to the dial 12 of the injection device 1.

The resolution of the sensing arrangement 200 is determined by the design of the injection device 1. A suitable angular resolution of the sensor arrangement 110 may be determined by Equation (1):

$$resolution = \frac{360 °}{units\_per \_rotation} \qquad ( 1 )$$

For instance, if one full rotation of the dial member 12 corresponds to a medicament dosage amount of 24 IU, then a suitable resolution for the rotation sensing arrangement 200 would be not more than 15°.

Typically, the angle of rotation of the dose dial 12 or dial ember measured by the rotation sensing arrangement 200 is proportional to the amount of medicament expelled. It is not necessary to determine a zero level or an absolute amount of medicament contained in the injection device 1. When the dose dial 12 rotates relative to the housing 10 during dose setting or expelling of a dose of the medicament the dose actually expelled can be precisely determined and monitored by the add-on device 100.

The add-on device 100 may comprises an interface 124 connected to the processor 240. The interface 240 may be a wireless communications interface for communicating with another external device 65, e.g. in form of a portable electronic device, via a wireless network such as Wi-Fi or Bluetooth®, or an interface for a wired communications link, such as a socket for receiving a Universal Series Bus (USB), mini-USB or micro-USB connector. For this, the interface 124 comprises a transceiver 126 configured for transmitting and receiving data. FIG. 3 depicts an example of an injection system in which the add-on device 100 is connected to an external electronic device 65, such as a personal computer 65, via a data connection 66 for data transfer. The data connection 66 may be of wired or wireless type.

For example, the processor 240 may store determined delivered medicament amounts and time stamps for the injections as they are administered by the user and subsequently, transfer that stored data to the external electronic device 65. The device 65 maintains a treatment log and/or forwards treatment history information to a remote location, for instance, for review by a medical professional.

The add-on device 100 or data collection device may be configured to store data such as delivered medicament amounts and time stamps of up to numerous injection events, such as 35 or more injection events. According to a once-daily injection therapy this would be sufficient to store a treatment history of about one month. Data storage is organized in a first-in first-out manner ensuring that most recent injection events are always present in the memory of the data collection device 100. Once transferred to an external electronic device 65 the injection event history in the add-on device 100 will be deleted. Alternatively, the data remains in the add-on device 100 and the oldest data is deleted automatically once new data is stored. This way the log in the data collection device is built up over time during usage and will always comprise the most recent injection events. Alternatively, other configuration could comprise a storage capacity of 70 (twice daily), 100 (three months) or any other suitable number of injection events depending on the therapy requirements and/or the preferences of the user.

In another embodiment, the interface 124 may be configured to transmit information using a wireless communications link and/or the processor 240 may be configured to transmit such information to the external electronic device 65 periodically.

The processor 240 may control the optional display 118 to show the determined medicament dose information, and/or to show an elapsed time since a last medicament dose was delivered. For example, the processor 240 may cause the display 118 to switch periodically between displaying the most recent determined medicament dosage information and the elapsed time.

The power source 120 may be a battery. The power source 120 may be a coin cell, or multiple coin cells arranged in series or parallel. A timer 115 may be also provided. In addition to, or instead of, switching the add-on device 100 on and off, the switch 122 may be arranged to trigger the timer 115 when engaged and/or disengaged. For example, if the timer 115 is triggered on both engagement or disengagement of the first and second electrical contacts of the switch or both operation and ceasing of operation of the switch 122, then the processor 240 may use the output from the timer 115 to determine a length of time during which the trigger 11 was pressed, for example to determine the duration of an injection.

Alternatively, or additionally, the processor 240 may use the timer 115 to monitor a length of time that has elapsed since an injection was completed, as indicated by a time of disengagement of respective switch components or ceasing of operation of the switch 122.

Optionally, the elapsed time may be shown on the display 118. Also optionally, when the switch 122 is next operated, the processor 240 may compare the elapsed time with a predetermined threshold, to determine whether a user may be attempting to administer another injection too soon after a previous injection and, if so, generate an alert such as an audible signal and/or a warning message on the display 118 or via the output 116. The output 160 may be configured to generate an audible sound or to induce a vibration hence to produce a tactile signal, e.g. for alerting the user.

### LIST OF REFERENCE NUMBERS

1 injection device
2 distal direction
3 proximal direction
4 dose incrementing direction
5 dose decrementing direction
6 cartridge
7 bung
8 drive mechanism
9 dose setting mechanism
10 housing
11 trigger
12 dose dial
13 dosage window
14 cartridge holder
15 injection needle
16 inner needle cap
17 outer needle cap
18 protective cap
19 protrusion
20 piston rod
21 bearing
22 first thread
23 pressure foot
24 second thread
25 barrel
26 seal
28 threaded socket
30 drive sleeve
31 threaded section
32 flange
33 flange
35 last dose limiter
36 shoulder
40 spring
41 recess
50 dose tracker
51 tracking stop feature
60 clutch
62 insert piece
64 stem
65 external device
66 data connection
80 number sleeve
81 groove
90 ratchet mechanism
91 ratchet feature
100 add-on device
101 housing
114 memory
115 timer
116 output
118 display
120 power source
122 switch
124 interface
126 transceiver
200 rotation sensing arrangement
201 first member
202 second member
203 axis of rotation
205 surface
206 surface
207 surface
208 surface
210 signal generator
220 sensor
230 interdigital electrode structure
231 first electrode
232 second electrode
233 electrode portion
234 electrode portion
235 electrode portion
236 electrode portion
237 electrode portion
238 electrode portion
240 processor
241 connecting portion
242 connecting portion
243 connector
244 connector
250 planar substrate
260 printed circuit board
270 electric field
280 magnetic field
290 ratchet arrangement
291 ratchet member
292 ratchet member
320 sensor
330 interdigital electrode structure
331 first electrode
332 second electrode
333 second electrode
420 sensor
422 strain gauge
430 interdigital electrode structure
431 first electrode
432 second electrode
433 meandering conductive structure
434 conductor section
436 conductor section

1.-16. (canceled)

17. A rotation sensing arrangement for an injection device, the rotation sensing arrangement comprising:
a first member and a second member, wherein the first member and the second member are rotatable relative to each other with regard to an axis of rotation;
at least one signal generator arranged on the first member;
at least one sensor arranged on the second member, wherein the at least one sensor comprises an interdigital electrode structure configured to generate an electrical signal in response to a movement of the at least one signal generator relative to the sensor; and
a processor connected to the at least one sensor and operable to calculate an angle of rotation of the first member relative to the second member on based on the electrical signal.

18. The rotation sensing arrangement of claim 17, further comprising a planar substrate, wherein the at least one sensor is arranged on the planar substrate.

19. The rotation sensing arrangement of claim 18, wherein the interdigital electrode structure is printed or coated on the planar substrate.

20. The rotation sensing arrangement of claim 17, further comprising a printed circuit board and wherein the interdigital electrode structure of the at least one sensor is arranged on the printed circuit board and wherein the processor is arranged on the printed circuit board.

21. The rotation sensing arrangement of claim 17, wherein the interdigital electrode structure is configured to generate an electric field and wherein the at least one signal generator is configured to modify the electric field.

22. The rotation sensing arrangement of claim 17, wherein the interdigital electrode structure comprises a first electrode and a second electrode, wherein the first electrode and the second electrode are arranged in an interleaved geometric configuration.

23. The rotation sensing arrangement of claim 17, wherein the interdigital electrode structure comprises a first electrode and a second electrode, wherein the first electrode and the second electrode are mutually electrically connected by a meandering conductive structure.

24. The rotation sensing arrangement of claim 17, wherein the signal generator comprises a signal generating portion made of a material having a relative permittivity larger than 3.

25. The rotation sensing arrangement of claim 17, wherein the interdigital electrode structure is configured to generate a magnetic field and wherein the at least one signal generator is configured to modify the magnetic field.

26. The rotation sensing arrangement of claim 17, wherein the at least one sensor is arranged at a predefined radial sensor distance from the axis of rotation and wherein the at least one signal generator is arranged at a predefined radial signal generator distance from the axis of rotation and wherein a difference between the radial sensor distance and the radial signal generator distance is smaller than or equal to a difference between a radial extent of the at least one sensor and a radial extent of the at least one signal generator.

27. The rotation sensing arrangement of claim 17, wherein a plurality of sensors of the at least one sensor is distributed across one side of the second member and/or wherein a plurality of signal generators of the at least one signal generator is distributed across one side of the first member facing towards the second member.

28. The rotation sensing arrangement of claim 17, wherein the at least one sensor and the at least one signal generator are permanently arranged out of mechanical contact.

29. The rotation sensing arrangement of claim 17, wherein the interdigital electrode structure is part of a strain gauge attached to the second member and wherein the interdigital electrode structure exhibits a measureable variation in electrical conductance in response to a flexible deformation of the second member.

30. The rotation sensing arrangement of claim 17, further comprising at least one ratchet arrangement engaged with at least one of the first member and the second member, wherein the ratchet arrangement is configured to support a rotation of the first member relative to the second member in discrete rotational steps.

31. An injection device for setting and expelling of a dose of a medicament, the injection device comprising:
a housing,
a trigger to initiate and/or to control expelling of the dose,
a dial member rotatable relative to the housing for setting of the dose, and
a rotation sensing arrangement comprising:
a first member and a second member, wherein the first member and the second member are rotatable relative to each other with regard to an axis of rotation, wherein the first member is rotationally locked to one of the dial member and the housing and wherein the second member is rotatable relative to the other one of the dial member and the housing,
at least one signal generator arranged on the first member,
at least one sensor arranged on the second member, wherein the at least one sensor comprises an interdigital electrode structure configured to generate an electrical signal in response to a movement of the at least one signal generator relative to the sensor, and
a processor connected to the at least one sensor and operable to calculate an angle of rotation of the first member relative to the second member based on the electrical signal.

32. The injection device of claim 31, further comprising a planar substrate, wherein the at least one sensor is arranged on the planar substrate and the interdigital electrode structure is printed or coated on the planar substrate.

33. The injection device of claim 31, further comprising a printed circuit board and wherein the interdigital electrode structure of the at least one sensor is arranged on the printed circuit board and wherein the processor is arranged on the printed circuit board.

34. A method for analyzing an operation of an injection device, the method comprising:
inducing a torque to one of a first member and a second member of the injection device relative to the other one of the first member and the second member thereby moving at least one signal generator relative to at least one sensor;
measuring an electrical signal of the interdigital electrode structure of the at least one sensor in response to a movement of the at least one signal generator relative to the at least one sensor; and
processing the electric signal by the processor to determine an angle of rotation of the first member relative to the second member based on the electrical signal.

35. The method of claim 34, further comprising:
detecting variations of a magnetic field generated by an interdigital electrode structure; and
triggering a modification of the magnetic field.

36. The method of claim 34, further comprising:
receiving signals from a plurality of sensors distributed across one side of the second member.
